(19) United States
(12) Patent Application Publication
(10) Pub. No.: US 2021/0253674 A1
(43) Pub. Date: Aug. 19, 2021

(54) ANTI-XYLAN SULFATE POLYCLONAL ANTISERUM AND USE THEREOF IN ELISA METHODS

(71) Applicant: Chemi S.P.A., Cinisello Balsamo (IT)

(72) Inventors: Flavio Leoni, Milano (IT); Giuliana Porro, Mediglia (MI) (IT)

(73) Assignee: Chemi S.P.A., Cinisello Balsamo (IT)

(21) Appl. No.: 17/252,612

(22) PCT Filed: Jul. 1, 2019
(86) PCT No.: PCT/IB2019/055557

§ 371 Date: Dec. 15, 2020

(30) Foreign Application Priority Data
Jul. 2, 2018 (IT) 102018000006849

(51) Int. Cl. C07K 16/06; G01N 33/543

(52) U.S. Cl. CPC C07K 16/06; G01N 2400/24; G01N 33/543

(57) ABSTRACT

The object of the present invention is an anti-xylan sulfate antiserum having an antibody titre of between 1/10000 and 1/15000, and obtainable using a specific immunization method, and use thereof in one or more ELISA methods for determining xylan sulfate levels in biological fluids.

The object of the present invention is an anti-xylan sulfate antiserum having an antibody titre of between 1/10000 and 1/15000, and obtainable using a specific immunization method, and use thereof in one or more ELISA methods for determining xylan sulfate levels in biological fluids.

### STATE OF THE ART

Xylan sulfate is a semi-synthetic polysaccharide polymer that has shown evidence of efficacy and good tolerability, and it is used as an anticoagulant, anti-inflammatory at the joint level and anti-inflammatory at urinary level, although its mechanism of action has not been elucidated.

To understand the mechanism of action of xylan sulfate, a better understanding of the pharmacological and pharmacodynamic properties that allow pharmacokinetic studies is needed. It is therefore important to have methods that allow the quantitative determination of xylan sulfate in biological liquids.

Currently, no validated chemical-physical analytical methods are available, and some studies have been reported in the literature wherein the analysis method, based on the use of monoclonal or polyclonal antibodies, showed poor sensitivity or low specificity issues.

To date, there are no anti-xylan sulfate monoclonal or polyclonal antibodies or ELISA kits for the determination of xylan sulfate in biological samples available in the market.

In J. Immunological Methods (1), a monoclonal antibody (MAb 5-B-10) capable of binding polysaccharides containing 2,3-, 2,6- and 4,6-disulfates esters substitutions on the pyranose ring, such as semisynthetic heparinoids, xylan sulfate, dextran sulfate, chondroitin sulfate E, galactose 2,6 disulfate carrageenan and glycosaminoglycan polysulfate, was developed.

Such antibody was used in ELISA assays to determine and quantify polysaccharide polysulfates in biological fluids, showing cross-reactivity with dextran sulfate and chondroitin sulfate, and a partial cross-reactivity with heparin, chondroitin sulfate A, C and D.

Therefore, the monoclonal antibody obtained in (1) shows specificity issues when used in ELISA assays to determine and quantify xylan sulfate in biological fluids.

In J. Immunological Methods (2), polyclonal antibodies were developed by intramuscular injection in rabbits, and these antibodies were used for developing ELISA methods. However, such polyclonal antibodies showed a low antibody titre (1/2000), cross-reactivity with heparin, and partial recognition of hyaluronic acid, xylan, heparan sulfate, chondroitin sulfate A and C, and dextran, thus reporting specificity issues for use in enzyme immunoassays for analysis and quantification of xylan sulfate in biological samples, rich in the components mentioned above.

In Journal of Urology (3), a polyclonal serum was developed following the immunization protocol of (2). The developed ELISA test showed interference with the glycoproteins present in the urine, and therefore the need for a proteolytic pre-treatment of the biological sample with Pronase, to eliminate the glycoproteins present.

In Xenobiotica (4), the pharmacokinetics and metabolic profiles of xylan sulfate were evaluated using radiolabeled xylan sulfate (³H-xylan sulfate) administered orally and using radiochromatographic techniques. With this method, poor absorption of xylan sulfate and low circulating radioactivity in the plasma were measured, precluding more extensive metabolic studies.

In Clinical Cancer Research (5), a radioimmunoassay (RIA) method for the determination of xylan sulfate in biological samples, based on the use of a polyclonal serum obtained by applying the immunization protocol of (2), was developed. This RIA method has the disadvantage of requiring the preparation of tyrosine-bound xylan sulfate and the subsequent labelling with ¹²⁵I, a procedure that could alter the structure of xylan sulfate, and therefore, its reactivity with the antiserum used. Furthermore, the use of radioactive material with the short half-life of the ¹²⁵I isotope leads to a limited use of the test over time.

It is therefore apparent the need to obtain antibodies having high titre and high specificity and sensitivity in order to develop ELISA methods that are able to specifically and accurately determine and quantify xylan sulfate levels in biological fluids, even in the presence of different glycosaminoglycans.

### Definitions

Unless otherwise defined, all terms of the art, notations, and other scientific terms used herein are intended to have the meanings commonly understood by those skilled in the art to which this description belongs. In some cases, terms with meanings that are commonly understood are defined herein for clarity and/or ready reference; therefore, the inclusion of such definitions herein should not be construed as being representative of a substantial difference with respect to what is generally understood in the art.

The terms “comprising”, “having”, “including”, and “containing” are to be construed as open-ended terms (i.e., meaning “comprising, but not limited to”), and are to be considered as a support also for terms such as “consist essentially of”, “consisting essentially of”, “consist of”, or “consisting of”.

The terms “consists essentially of”, “consisting essentially of” are to be construed as semi-closed terms, meaning that no other ingredients which materially affects the novel characteristics of the invention are included (optional excipients may therefore be included).

The terms “consists of”, “consisting of” are to be construed as closed terms.

The acronym “ELISA” means enzyme-linked immunosorbent assay.

The term “competitive ELISA” means an enzyme immunoassay wherein the antigen-specific antibodies present in the antiserum compete, for their binding to the antigen fixed on a plastic support, with the antigen present in solution in the analyzed biological sample. It follows that the greater the amount of antigen in solution in the biological sample, the lower the binding with the antigen fixed on the plastic support and, consequently, the smaller the developed signal.

The term “non-competitive ELISA” or “direct ELISA” means an enzyme immunoassay wherein the antigen-specific antibodies present in the antiserum bind to the antigen present in solution in the analyzed biological sample, which was previously adsorbed onto the plastic support. It follows that the greater the amount of antigen in solution in the biological sample, the greater the amount of antigen that adsorbs onto the plastic support and, consequently, the greater is the developed signal.

The term “antibody titre” means the reciprocal of the lowest concentration (or highest dilution) of the animal serum that maintains 50% of the maximum detectable activity against a known antigen.

### DESCRIPTION OF THE FIGURES

FIG. 1. Titration of the anti-xylan sulfate polyclonal serum from Rabbit C by competitive ELISA, vs. xylan, chondroitin sulfate and xylan sulfate.

FIG. 2. Titration of the anti-xylan sulfate polyclonal serum from Rabbit C by direct ELISA and antibody titre thereof.

FIG. 3. Anti-xylan sulfate antibody response at day 104 in the immunized rabbits, measured by direct ELISA.

FIG. 4. Specificity of the anti-xylan sulfate antibody response from rabbit C.

FIG. 5. Titration of the anti-xylan sulfate polyclonal serum from Rabbit C by direct ELISA.

FIG. 6. Titration of the anti-xylan sulfate polyclonal serum from Rabbit C by competitive ELISA.

FIG. 7. Titration of xylan sulfate in rat urine by direct ELISA.

FIG. 8. Titration of xylan sulfate in human urine from a healthy donor by direct ELISA.

FIG. 9. Titration of xylan sulfate in human urine samples from healthy donor and from a vegetarian healthy donor by direct ELISA.

FIG. 10. Titration of xylan sulfate in samples of human plasma at different dilutions and human urine from healthy donors by direct ELISA.

### DESCRIPTION OF THE INVENTION

It has surprisingly been observed that the polyclonal antiserum obtained by subcutaneous immunization in the rabbit using an immunization protocol lasting for at least 100 days, has a significantly higher antibody titre than the antibody titre obtained with other, shorter immunization methods, and allows to develop particularly sensitive competitive and non-competitive ELISA assays for detection and/or quantification of xylan sulfate levels in biological fluids.

A clear advantage of the present invention is the greater specificity of the antibody obtained when compared to what previously reported in the literature, thus overcoming the issues encountered using the methods described in the known art.

On the basis of specific knowledge in the field, it would not have been possible to foresee that the application of the immunization protocol shown herein would have led to such a marked specificity improvement effect.

The obtaining of a high titre antiserum is an important aspect for the development of immunochemical assays, since the higher the titre, the greater the reactivity towards the antigen, with consequent lower non-specific binding and an improved signal-to-noise ratio of the assay.

Furthermore, the greater the antibody titre, the greater the dilution to be used in the test and, consequently, the lower the amount of antiserum required for the analysis of each sample and the greater the number of samples that can be analyzed.

The possibility of analyzing a large number of samples with the same antiserum has the advantage of reducing variability in the results of sample analysis obtained in studies performed at different times.

An object of the present invention is therefore an anti-xylan sulfate polyclonal antiserum having an antibody titre of between 1/10000-1/15000.

According to a preferred aspect, the anti-xylan sulfate polyclonal antiserum having an antibody titre of between 1/10000-1/15000 is obtained by the following steps:

a) one or more subcutaneous injection of xylan sulfate antigen complexed with methyl-BSA and emulsified with complete (CFA) or incomplete (IFA) Freund's adjuvant into a mammalian;

b) collecting said polyclonal antiserum.

According to the present invention, the first injection is performed with complete Freund's adjuvant, while the subsequent injections are performed with Freund's incomplete adjuvant.

In a further preferred aspect, said anti-xylan sulfate polyclonal antiserum is characterized in that it does not recognize unfractionated heparin (UFH), low molecular weight heparin (LMW), xylan, dextran sulfate, and glycosaminoglycans selected from heparan sulfate and chondroitin sulfate A, B, C, D, or E.

Preferably said mammal is a rabbit.

According to a preferred aspect, said polyclonal antiserum is characterized in that step a) provides for the use of 0.5-1.5 mg of antigen/injection, preferably 1 mg of antigen/injection.

According to a further preferred aspect, said polyclonal antiserum is characterized in that, in step a), 5-10 subsequent injections, for a maximum period of 100-110 days, preferably 104 days, are performed.

According to a further preferred aspect, in step a) according to the present invention, injections subsequent to the first one are performed at intervals of 4-40 days, preferably from 7 to 14 days.

According to a further preferred aspect, the aforementioned polyclonal antiserum is characterized in that, in step b), the polyclonal antiserum is isolated from the mammalian after at least 100-110 days, preferably 104 days.

According to a further preferred aspect, the immunological reactivity of the aforementioned polyclonal antiserum is due only to antibodies of the IgG subclass.

According to a further aspect, the polyclonal antiserum according to the present invention is used for the development of ELISA methods for identification and/or quantification of xylan sulfate levels in biological fluids.

Preferably, said biological fluids are selected from serum, plasma and urine.

Said ELISA method can be a direct or non-competitive ELISA method, or a competitive ELISA method.

The direct or non-competitive ELISA method according to the present invention is characterized by the following steps:
a) immobilization of xylan sulfate present in the biological sample, or in titrated standard solutions, on a pre-treated solid support, through incubation for 12-20 hours at room temperature;
b) blocking the aspecific binding sites by incubating with PBS containing 10% fetal calf serum (FCS) for 60 minutes at room temperature;
c) incubation with polyclonal antiserum according to the present invention;
d) incubation with a rabbit IgG-specific antibody conjugated to an enzyme for 90 minutes at room temperature;
e) incubation with a solution containing the enzymatic substrate for 5-10 minutes at room temperature;
f) blocking the enzymatic reaction by addition of a denaturing solution, preferably 2N sulfuric acid;
g) measuring the optical density of the solution;
h) calculating xylan sulfate concentration in the biological samples.

Preferably, the calculation of xylan sulfate contained in the biological samples is performed by non-linear regression vs. values of the samples in the standard curve.

According to a preferred aspect, the polyclonal antiserum is used at dilutions of between 1:10000 and 1:1000, more preferably 1:5000.

Preferably, the rabbit IgG-specific antibody conjugated to the enzyme is used at a dilution of between 1:3000 and 1:5000, preferably 1:3000.

Preferably, the enzymatic substrate (TMB) is used in a 1:1 solution of 0.4 mg/mL TMB and 0.02% H₂O₂.

Preferably, the denaturing solution is used at 2N concentration.

According to a preferred aspect, the enzyme conjugated to the IgG antibody of point d) is horseradish peroxidase or alkaline phosphatase.

According to a further preferred aspect, following steps b, c, d and e, a washing cycle of the wells with washing buffer is performed.

Preferably, the measurement of the optical density of the solution is performed by a spectrophotometer for microplates, at a wavelength specific for the enzymatic reaction product, preferably said wavelength is selected from 450 nm, 495 nm and 405 nm.

Preferably, said ELISA method is characterized in that the intensity of the color developed in point g) is directly proportional to the amount of xylan sulfate.

For the development of ELISA methods according to the present invention, a batch of xylan sulfate is selected as the reference sample (standard) and is used to prepare the standard curves.

Preferably, 7 concentrations of the standard and 3 concentrations of the sample to be tested are prepared, by preparing at least 3 replicates for each concentration of the standard and for each concentration of the sample to be tested, generally by subsequent 1:2 dilutions of the stock solutions.

The reference sample concentrations used for the standard curve generally range from 1 ng/mL to 10000 ng/mL.

Preferably, the following reference sample concentrations are used: 4, 12, 37, 111, 333, 1000 and 3000 ng/mL.

The competitive ELISA method according to the present invention is characterized by the following steps:
a) immobilization of xylan sulfate on a pre-treated solid support, through incubation for 12-20 hours at room temperature;
b) blocking the aspecific binding sites by incubating with PBS containing 10% FCS for 60 minutes at room temperature;
c) incubation with polyclonal antiserum with a biological sample containing xylan sulfate, or with titrated solutions of xylan sulfate, for 90 minutes at room temperature;
d) incubation with a rabbit IgG-specific antibody conjugated to an enzyme for 90 minutes at room temperature;
e) incubation with a solution containing the enzymatic substrate for 5-10 minutes at room temperature;
f) blocking the enzymatic reaction by addition of a denaturing solution, preferably 2N sulfuric acid;
g) measuring the optical density of the solution;
h) calculating xylan sulfate concentration in the biological samples.

Preferably, the calculation of the concentration of xylan sulfate contained in the biological samples is performed by non-linear regression vs. values of the samples in the titrated solution.

An advantage of the competitive ELISA method is that this method also allows the measurement of xylan sulfate metabolites that may not bind to poly-lysine but be nevertheless recognized by the antiserum when present in a biological sample.

According to a preferred aspect, following the steps a, b, c, d and e, a washing cycle of the wells is carried out with washing buffer.

Preferably the rabbit IgG-specific antibody conjugated to the enzyme is used at a dilution of between 1:3000 and 1:5000, preferably 1:3000.

Preferably the enzymatic substrate (TMB) is used in a 1:1 solution of 0.4 mg/mL TMB and 0.02% H₂O₂.

Preferably, the denaturing solution is used at 2N dilution.

According to a further preferred aspect, the polyclonal antiserum used in step c) is used in a fixed concentration, preferably at a concentration of 1:10000.

Preferably, the measurement of the optical density of the solution is measured through a spectrophotometer by plates, at a specific wavelength for the product of the enzymatic reaction, preferably said wavelength is selected from 405, 450 and 540 nm.

According to a preferred aspect, the amount of xylan sulfate used in step a) is of between 0.1 μg/well and 1 μg/well, preferably 0.1 μg/well.

Preferably, said competitive ELISA method is characterized in that the intensity of the color developed in step g) is inversely proportional to xylan sulfate amount.

Preferably, the washing buffer used in the ELISA methods of the present invention is phosphate buffer, more preferably this buffer is PBS+0.05% Tween 20.

In a further preferred aspect, the washing buffer used in the methods of the present invention comprises a mixture of phosphate buffer having a pH of between 6.5 and 8.0 with a molarity of between 50 and 200 nM, and Tween 20 in the range of between 0.01% and 0.1%.

According to a further preferred aspect, the solid support used in the ELISA methods of the present invention is treated with poly-lysine, polyvinyl sulfonate or allylamine:octadiene.

Preferably, such solid support consists of a material selected from polycarbonate, polystyrene, polypropylene, polyethylene, glass, cellulose, nitrocellulose, silica gel, or polyvinyl chloride, more preferably polystyrene.

Preferably, such solid support is selected from 96-well ELISA plates.

Preferably, the substrate dye according to the present invention is p-nitrophenylphosphate, hydrogen peroxide, o-phenylenediamine or 3,3′,5,5′-tetramethylbenzidine (TMB).

Preferably, the enzyme used in the ELISA methods of the present invention is selected from horseradish peroxidase or alkaline phosphatase.

Preferably, the blocking agent is selected from bovine serum albumin, fetal calf serum, gelatin or low-fat powdered milk.

Data processing according to the present invention involves a non-linear regression of the inhibition values in the standard curve consisting of at least 6 different concentrations. Preferably, a 4-parameter logistic regression, performed by specialized software (for example GraphPad Prism) is used in the ELISA methods of the present invention.

All samples are analyzed in 3 replicates. Their reactivity is expressed as IC₅₀, i.e. the concentration of the sample determining 50% inhibition of the antiserum maximum binding.

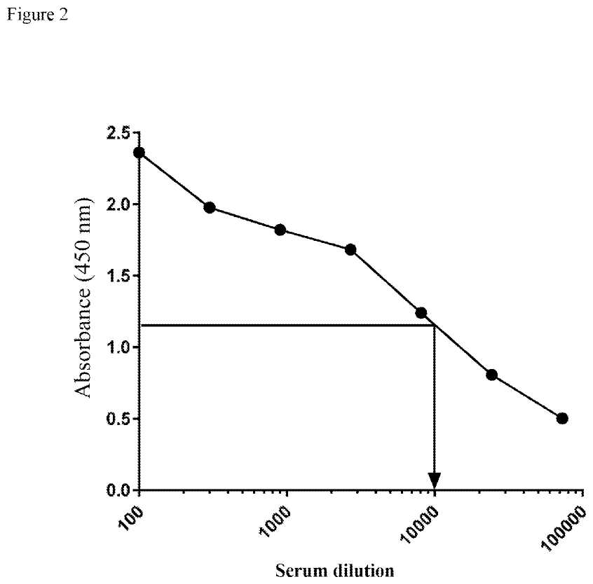

Based on the results obtained in the experimental data section it can be concluded that:
the immunization protocol used in the present invention (see below, Table 1) allows to obtain a more reactive antiserum than the antisera obtained from animals immunized intramuscularly.
moreover, the subcutaneous route allows a better tolerability of the antigen emulsion based on Freund's complete adjuvant, therefore, it is, also from an ethical point of view, the preferred way to induce lesser suffering of the animal.
the number and frequency of the antigen boosts used are more effective in obtaining a polyclonal antiserum as the xylan sulfate antigen has generally proved to be a low immunogenic antigen;
the polyclonal antiserum obtained is highly specific for xylan sulfate and has an antibody titre of between 1/10000 and 1/15000 (FIG. 2), i.e. 5-8 times greater than the titre of polyclonal or monoclonal antibodies available in the literature; this characteristic allows to have an antiserum with high reactivity to the antigen and a negligible binding to molecules structurally different compared to the antigen, resulting in better sensitivity and an analytical method with a wider dynamic range than the;
such polyclonal antiserum shows no cross-reaction with heparin and with a whole series of glycosaminoglycans (see Table 3), therefore this antiserum has a greater specificity than the monoclonal and polyclonal anti-xylan sulfate antibodies described in the literature up to now;
the direct and competitive ELISA tests, developed using this polyclonal antiserum, resulted to be highly specific and sensitive in quantifying the presence of xylan sulfate in different biological samples, such as serum, plasma and urine;
the sensitivity and accuracy of the ELISA assays developed with such antiserum are suitable for measuring the expected levels of xylan sulfate;
the ELISA methods developed allow quantification of xylan sulfate both in human plasma samples and human urine samples without needing a pre-treatment of the biological sample;
the ELISA methods developed allow the quantification of xylan sulfate in rat urine samples, resulting therefore suitable for bio-equivalence studies;
the competitive ELISA method allows the determination of xylan sulfate metabolites that may not bind to poly-lysine and, therefore, are not measurable by the direct ELISA method, despite having one or more epitopes recognized by the anti-xylan sulfate polyclonal serum.

Below are some examples whose nature does not limit the applicability of the invention.

### Materials and Methods

### Example 1

### Production of Polyclonal Antibodies

Five New Zealand White rabbits were treated, and the xylan sulfate used, complexed with methyl-BSA, was extracted “in house” from capsules of product for pharmaceutical use.

The contents of the capsules was poured into a beaker to which demineralized water was added. The suspension thus obtained was left under stirring overnight at room temperature. In the morning, the suspension was filtered under vacuum using a cellulose acetate filter and the resulting solution was then lyophilized. Identity and quality of the product obtained were evaluated by water content analysis (Karl Fisher), NMR studies and molecular weight distribution.

The antigen preparation procedure was performed as follows.

1% methyl-BSA solution was slowly added to 1% xylan sulfate solution in water, under stirring at room temperature. After 30 minutes, the aggregate formed was separated by centrifugation (2000 rpm for 20 min), washed with PBS (1.5 mM phosphate, 0.1 M NaCl, pH 7.2) and resuspended in the same buffer at a 2 mg/mL concentration. The solution was then divided into aliquots which were kept at −20° C. until use.

The immunization was performed using xylan sulfate complexed with mBSA and emulsified in complete Freund's adjuvant (CFA) or incomplete Freund's adjuvant (IFA), at a dose of 1 mg/rabbit as shown in Table 1.

TABLE 1
Immunization protocol with xylan sulfate in the rabbit.
Immunization protocol

| | |
|---|---|
| Day 0 | 1 mg xylan sulfate-mBSA/rabbit |
| First antigen injection | |
| Day 21 | 1 mg xylan sulfate-mBSA in |
| Antigen boost | IFA/rabbit |
| Day 35 | 1 mg xylan sulfate-mBSA in |
| Antigen boost | IFA/rabbit |
| Day 74 | 1 mg xylan sulfate-mBSA in |
| Antigen boost | IFA/rabbit |
| Day 81 | 1 mg xylan sulfate-mBSA in |
| Antigen boost | IFA/rabbit |
| Day 92 | 1 mg xylan sulfate-mBSA in |
| Antigen boost | IFA/rabbit |
| Day 96 | 1 mg xylan sulfate-mBSA in |
| Antigen boost | IFA/rabbit |
| Day 104 | Serum collection |
| Sacrifice | |

Two rabbits were treated intramuscularly with xylan sulfate-mBSA mixed with IFA while, based on previous experience, three rabbits were treated subcutaneously with xylan sulfate-mBSA mixed with CFA.

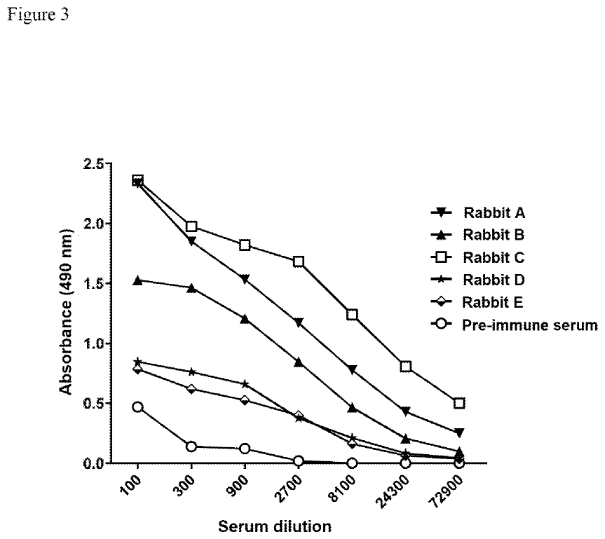

The antibody reactivity of each animal at the end of the study is reported in Table 2 and FIG. 3.

TABLE 2
Anti-xylan sulfate antibody response of immunized
rabbits at day 104, measured by “direct ”ELISA ”.

| Serum | Pre-immune | Rabbit | Rabbit | Rabbit | Rabbit | Rabbit |
|---|---|---|---|---|---|---|
| Dilution | Serum | A | B | C | D | E |
| 1:100 | 0.470* | 2.335 | 1.526 | 2.362 | 0.846 | 0.785 |
| 1:300 | 0.139 | 1.849 | 1.462 | 1.975 | 0.676 | 0.620 |
| 1:900 | 0.121 | 1.532 | 1.206 | 1.820 | 0.659 | 0.526 |
| 1:2700 | 0.019 | 1.169 | 0.845 | 1.682 | 0.376 | 0.399 |
| 1:8100 | 0.000 | 0.776 | 0.465 | 1.238 | 0.210 | 0.162 |
| 1:24300 | 0.000 | 0.430 | 0.206 | 0.806 | 0.083 | 0.064 |
| 1:72900 | 0.000 | 0.248 | 0.099 | 0.502 | 0.043 | 0.037 |

*Absorbance (450 nm)

Based on the immune sera titrations reported above, it was decided to use the serum from rabbit C for subsequent studies.

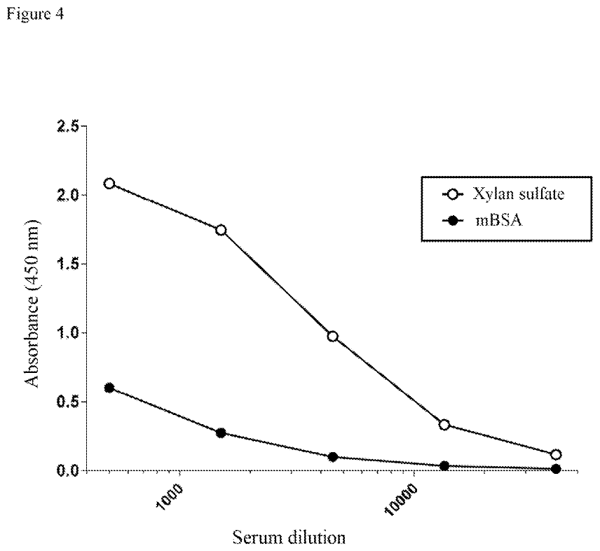

In order to better characterize the selected polyclonal serum, the specificity for xylan sulfate was evaluated by measuring the reactivity vs. both xylan sulfate and mBSA, used as a carrier, in direct ELISA. The results obtained are shown in FIG. 4.

The reported results show a good specificity for the polyclonal serum of rabbit C towards xylan sulfate vs. mBSA.

### Example 2

### Development of Direct or Non-Competitive ELISA Test

ELISA tests reported in the literature (1, 2, 3) are based on immobilization of xylan sulfate on poly-lysine coated plates.

After a few attempts at preparing the poly-lysine coated plates (based on the scarce literature information) which showed a great variability (determined as ELISA vs. xylan sulfate), it was decided to use commercial poly-lysine coated plates.

Such plates were used in all the following experiments.

The developed direct ELISA test is summarized below in its various steps:
1. Binding of xylan sulfate (standard curve: 4.1, 12.3, 37, 111, 333, 1000 and 3000 ng/mL) and xylan sulfate containing samples (100 μL/well) to poly-lysine coated plates (at room temperature overnight, 14-20 hours)
2. Three washing cycles (300 μL/well) with PBS using an automatic plate washer
3. Blocking the aspecific binding sites with 300 μL/well of PBS containing 10% FCS (60 min at room temperature)
4. Three washing cycles (as in point 2) with PBS using an automatic plate washer
5. Incubation with anti-xylan sulfate polyclonal serum (100 μL/well at the dilution of 1:5000) for 90 min at room temperature under shaking.
6. Three washing cycles (as in point 2) with PBS using an automatic plate washer
7. Incubation with 100 μL/well of a commercial anti-rabbit IgG-HRP antibody (at the dilution of 1:3000) for 90 min at room temperature under shaking
8. Three washing cycles (as in point 2) with PBS using an automatic plate washer
9. Addition of TMB substrate (1:1 solution of TMB 0.4 mg/mL and 0.02% H₂O₂, 100 μL/well) for 7 min at room temperature in the dark
10. Blocking the reaction with 2N sulfuric acid (50 μL/well)
11. Measuring absorbance (450 nm) by microplate spectrophotometer
12. Calculating xylan sulfate concentration in the biological sample by non-linear regression of the standard curve

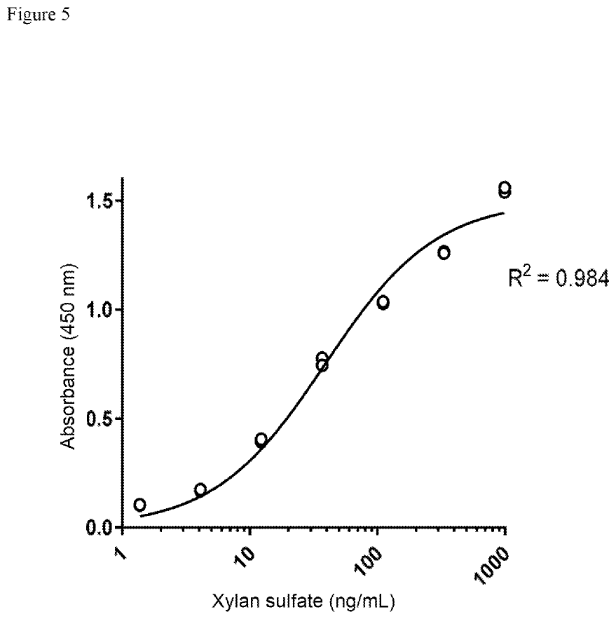

Titration of polyclonal serum from Rabbit C vs. xylan sulfate performed with the optimized ELISA test is shown in FIG. 5.

The titration curve (4.1, 12.3, 37, 111, 333, 1000 and 3000 ng/mL), obtained by non-linear regression, shows a quantitative response from 2-5 ng/mL to about 1 μg/mL of xylan sulfate (in phosphate buffer).

The test is therefore accurate in a xylan sulfate concentration range of between 2 ng/mL and 1 μg/mL.

### Example 3

### Development of Competitive ELISA Test

ELISA tests reported in the literature (1, 2, 3) are of a competitive type, i.e. the test evaluates the ability of xylan sulfate in solution to inhibit the polyclonal antiserum binding to xylan sulfate immobilized on the poly-lysine coated plates.

The developed competitive ELISA test is summarized below in its various steps:
1. Binding of xylan sulfate (100 μL/well) to poly-lysine coated plates (at room temperature overnight, 14-20 hours)
2. Three washing cycles (300 μL/well) with PBS using an automatic plate washer
3. Blocking the aspecific binding sites with 300 μL/well of PBS containing 10% FCS (60 min at room temperature)
4. Three washing cycles (300 μL/well) with PBS using an automatic plate washer
5. Co-incubation of anti-xylan sulfate polyclonal antiserum (50 μL/well at the dilution of 1:5000) with 50 μL of the biological sample containing xylan sulfate or with titrated xylan sulfate (standard curve: 5.2, 25.6, 128, 640, 3200 and 16000 ng/mL for 90 min at room temperature under shaking).
6. Three washing cycles (as in point 2) with PBS using an automatic plate washer
7. Incubation with 100 μL/well of a commercial anti-rabbit IgG-HRP antibody (at the dilution of 1:3000) for 90 min at room temperature under shaking
8. Three washing cycles (as in point 2) with PBS using an automatic plate washer
9. Addition of 100 μL/well of substrate (1:1 solution of TMB 0.4 mg/mL and 0.02% H₂O₂) for 7 min at room temperature in the dark
10. Blocking the reaction with 50 μL/well of 2N sulfuric acid
11. Measuring absorbance (450 nm) by microplate spectrophotometer
12. Calculating xylan sulfate concentration in the biological sample by non-linear regression of the standard curve

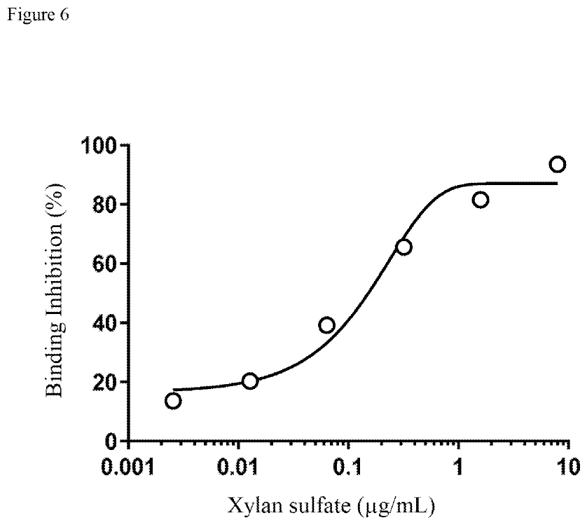

Titration of polyclonal serum from Rabbit C vs. xylan sulfate performed with such an optimized competitive ELISA test is reported in FIG. 6.

The test is therefore accurate in a xylan sulfate concentration range of between 1 ng/mL and 10 μg/mL.

### Example 4

Reactivity of the Polyclonal Antiserum Vs. Different Glycosaminoglycans or Negatively Charged Compounds (GAG, Heparins aDNA)

Since the binding to poly-lysine of the different glycosaminoglycans and negatively charged compounds under test is uncertain, the competitive ELISA test was used to determine the polyclonal antiserum specificity vs. compounds chemically similar to xylan sulfate.

13 molecules were analyzed to which the obtained antiserum could be reactive, and the reactivity of each of them with the antiserum was evaluated in comparison with commercial xylan sulfate. The reactivity of each compound was determined as IC₅₀ (concentration of the compound determining 50% inhibition of the maximum antiserum binding) based on the titration curve analyzed by non-linear regression.

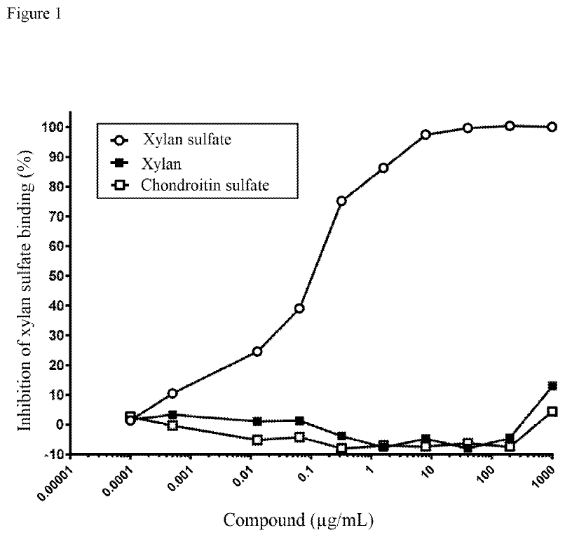

FIG. 1 shows titrations of two negative compounds (xylan and chondroitin sulfate) with serum from Rabbit C compared to titration of xylan sulfate.

The overall results obtained are summarized in the following table.

TABLE 3
Reactivity (IC₅₀) vs. different glycosaminoglycans of anti-xylan
sulfate polyclonal antiserum from Rabbit C of Example 1.

| Compound | IC₅₀ (μg/mL) |
|---|---|
| Xylan Sulfate | 0.122 |
| Heparin (UFH) | >1000 |
| Enoxaparin | >1000 |
| Heparan Sulfate | >1000 |
| Chondroitin Sulfate | >1000 |
| Chondroitin Sulfate A | >1000 |
| Chondroitin Sulfate C | >1000 |
| Chondroitin Sulfate E | >1000 |
| Oversulfated Chondroitin Sulfate | 1.014 |
| Dermatan Sulfate | >1000 |
| Keratan Sulfate | >1000 |
| Xylan | >1000 |
| Dextran Sulfate | 19.7 |
| DNA | >1000 |

The results obtained clearly indicate that 11 of the 13 compounds analyzed are not recognized by the polyclonal serum (IC₅₀>1000 μg/mL). Only oversulfated chondroitin sulfate and dextran sulfate show a slight reactivity (IC₅₀ of 1.014 μg/mL and 19.7 μg/mL, respectively), but significantly lower compared to xylan sulfate, for which the IC₅₀ value (0.122 μg/mL) is significantly lower.

These data confirm the good specificity of the produced polyclonal anti-serum (as summarized in Table 4) which is more selective when compared to the polyclonal serum produced in (2), which shows a strong cross-reaction with unfractionated heparin, MAb 5-B-10 produced in (1), which reacts with dextran sulfate, several chondroitin sulfates (A, B, C, D) and, in particular, with chondroitin sulfate E and with heparin UFH, and the polyclonal serum from (3) that showed interference with glycoproteins present in patients' urine.

TABLE 4
Reactivity vs. different compounds of anti-xylan sulfate polyclonal
antiserum from Rabbit C of Example 1 in comparison with polyclonal
serum from (2) and MAb 5-B-10 of (1).

| | Polyclonal | | |
|---|---|---|---|
| | Serum from | Polyclonal | MAb |
| | Rabbit C of | Serum | 5-B-10 |
| Compound | Example 1 | from (2) | from (1) |
| Heparin UFH | − | +++ | + |
| Heparin LMW | − | n.d.* | n.d. |
| Heparan Sulfate | − | − | − |
| Hyaluronic Acid | n.d. | − | n.d. |
| Xylan | − | − | − |
| Chondroitin Sulfate A | − | − | + |
| Chondroitin Sulfate B | n.d. | n.d. | +/− |
| Chondroitin Sulfate C | − | − | + |
| Chondroitin Sulfate D | n.d. | n.d. | + |
| Chondroitin Sulfate E | − | n.d. | +++ |
| Oversulfated | +/− | n.d. | n.d. |
| Chondroitin Sulfate | | | |
| Dextran Sulfate | − | − | +++ |

*n.d.: not determined.

### Example 5

### Analysis of Xylan Sulfate Concentration in Rat and Human Urine, and in Human Plasma.

The development of an ELISA test is needed to quantify the presence of xylan sulfate in biological fluids.

The animal study involves the evaluation of xylan sulfate levels present in both urine and plasma. In particular, xylan sulfate levels in urine are of particular importance given the therapeutic target of the drug.

Several experiments with rat urine samples supplemented with xylan sulfate and varying the experimental conditions (dilution of the urine sample and dilution of the polyclonal serum) were performed in order to obtain a reliable measurement of xylan sulfate levels.

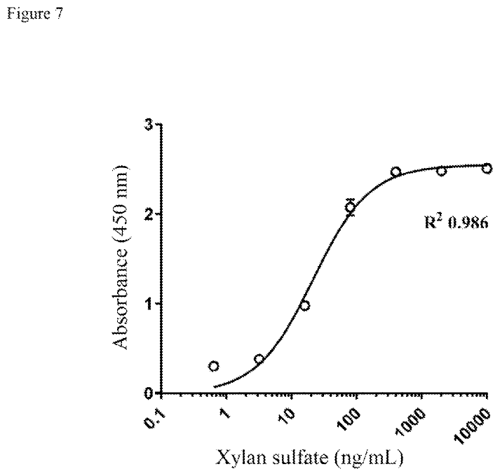

FIG. 7 shows the titration of xylan sulfate dissolved in rat urine diluted 1:3 with phosphate buffer and the polyclonal serum diluted 1:1000.

The results indicate that the direct ELISA test on rat urine samples has a good sensitivity (about 5 ng/mL) and a good linearity up to concentrations of about 300 ng/mL.

In a subsequent experiment, xylan sulfate “recovery”, i.e. the xylan sulfate concentration measured vs. the expected concentration, was evaluated by analyzing three replicates of rat urine samples supplemented with known amounts of xylan sulfate in a concentration range from 10 to 100 ng/mL.

The recovery of three replicates is shown in the following table.

TABLE 5
Evaluation of the recovery of three different series of xylan sulfate serial
samples in rat urine by direct ELISA.

| Expected | | | | | | | |
|---|---|---|---|---|---|---|---|
| concentration | | | | | | | |
| measured | Xylan | | Xylan | | Xylan | | |
| of xylan | sulfate | | sulfate | | sulfate | | Mean |
| sulfate | measured | Recovery | measured | Recovery | measured | Recovery | Recovery |
| (ng/mL) | Replicate 1 | (%) | Replicate 2 | (%) | Replicate 3 | (%) | (%) |
| 100 | 99.2 | 99.2 | 94.9 | 94.9 | 120.4 | 120.4 | 105 |
| 75 | 76.9 | 102.5 | 74.8 | 99.8 | 93.7 | 124.9 | 109 |
| 50 | 48.7 | 97.3 | 40.3 | 80.6 | 49.8 | 99.7 | 94 |
| 43 | 45.3 | 105.3 | 38.5 | 89.5 | 46.8 | 108.9 | 101 |
| 37 | 33.4 | 90.3 | 33.2 | 89.8 | 37.7 | 101.8 | 94 |
| 30 | 23.5 | 78.3 | 24.9 | 82.9 | 25.5 | 84.9 | 82 |
| 22 | 11.3 | 51.2 | 14 | 63.5 | 16.7 | 76.0 | 64 |
| 10 | 5.7 | 57.4 | 5.7 | 57.1 | 6.9 | 68.6 | 61 |

Xylan sulfate recovery is optimal (between 80 and 110%) in the range of doses between 30 and 100 ng/mL, while at the lower doses (range between 10 and 22 ng/mL) it is lower and equal to about 60%.

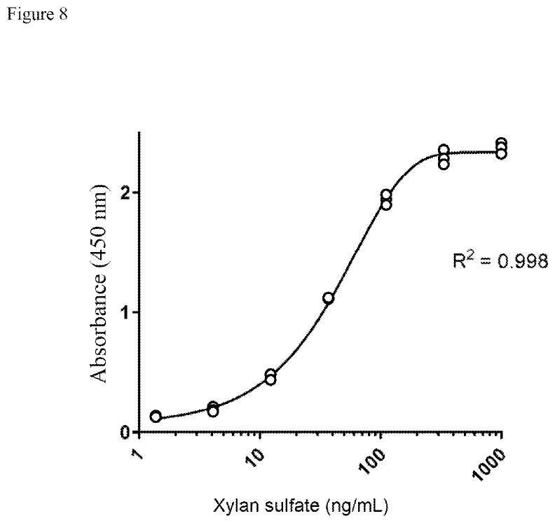

The ELISA test was therefore used with samples of human urine from a healthy donor and a representative titration curve is shown in FIG. 8.

In a subsequent test, we compared the titration of xylan sulfate dissolved in human urine from a healthy donor to the titration of xylan sulfate in human urine from a vegetarian subject, who could have glycosaminoglycans interfering with the assay.

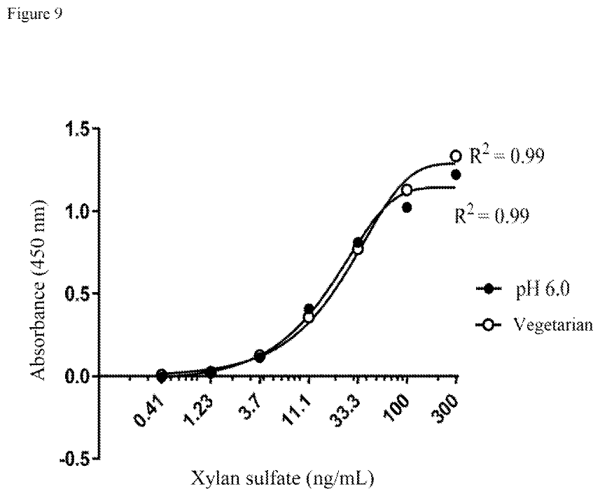

The results obtained, reported in FIG. 9, indicate that the test response is superimposable for both urine samples, confirming the assay specificity.

In a subsequent experiment, xylan sulfate “recovery” was evaluated by analyzing three replicates of human urine samples supplemented with known amounts of xylan sulfate in a concentration range from 0.7 to 120 ng/mL.

The calibration line of xylan sulfate in human urine is reported in FIG. 8, while the recovery of the three replicates is reported in the following table.

TABLE 6
Evaluation of the recovery of three different series of xylan sulfate serial
samples in human urine by direct ELISA.

| Expected | | | | | | | |
|---|---|---|---|---|---|---|---|
| concentration | | | | | | | |
| measured | Xylan | | Xylan | | Xylan | | |
| of xylan | sulfate | | sulfate | | sulfate | | Mean |
| sulfate | measured | Recovery | measured | Recovery | measured | Recovery | Recovery |
| (ng/mL) | Replicate 1 | (%) | Replicate 2 | (%) | Replicate 3 | (%) | (%) |
| 120 | 99.84 | 83.20 | 99.69 | 83.08 | 101.73 | 84.77 | 84 |
| 75 | 64.03 | 85.37 | 79.71 | 106.28 | 76.75 | 102.33 | 98 |
| 43 | 57.47 | 133.65 | 51.89 | 120.68 | 58.09 | 135.10 | 130 |
| 22 | 23.72 | 107.84 | 25.09 | 114.03 | 20.51 | 93.22 | 105 |
| 5 | 3.60 | 71.90 | 3.57 | 71.36 | 3.89 | 77.89 | 74 |
| 0.7 | 1.49 | 212.59 | 1.46 | 208.85 | 1.80 | 257.61 | 226 |

The recovery of xylan sulfate is optimal (between 85 and 130%) in the range of doses between 22 and 100 ng/mL, while at a dose of 5 ng/mL it drops to 74%. As expected, at the lowest dose (0.7 ng/mL), outside the titration curve, the recovery is overestimated and unreliable.

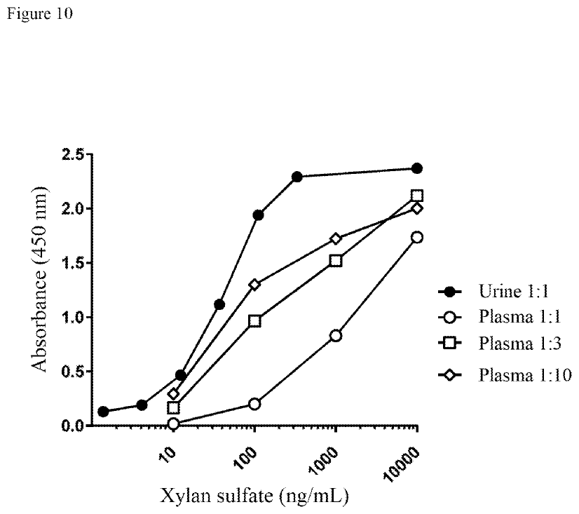

The direct ELISA test was also used for the analysis of xylan sulfate levels in human plasma. At the moment, only preliminary data are available that indicate the use of this test also for these samples is feasible. The titration of xylan sulfate in human plasma is reported in FIG. 10.

Human plasma samples containing xylan sulfate, when diluted at least 1:3, show a titration curve similar to that obtained with urine. In such a condition, the test shows a sensitivity of about 10 ng/mL.

Overall, the results obtained with urine samples, both rat and human, indicate that the direct ELISA test enables the evaluation of xylan sulfate levels in a reliable manner. Furthermore, the test is particularly sensitive for measuring the expected levels of xylan sulfate, in a concentration range from 2 ng/mL to 1 μg/mL in the direct ELISA test and in a concentration range from 1 ng/mL to 10 μg/mL in the competitive ELISA test.

### REFERENCES

1. Immunological Methods, 126 (1990) 39-49.
2. J. Immunological Methods, 166 (1991) 53-59.
3. J. Urol., 175 (3 Pt 1) (2006) 1143-1147.
4. Xenobiotica 35(8) (2005) 775-784.
5. Clinical Cancer Res. 3 (1997) 2347-2354.

1. Anti-xylan sulfate polyclonal antiserum having an antibody titre of between 1/10000 and 1/15000.

2. Anti-xylan sulfate polyclonal antiserum according to claim 1 obtainable by the following steps:
a) performing one or more subcutaneous injection of xylan sulfate antigen complexed with methyl-BSA and emulsified with complete or incomplete Freund's adjuvant into a mammalian; and
b) collecting of said polyclonal antiserum.

3. Polyclonal antiserum according to claim 1, wherein said polyclonal antiserum does not recognize unfractionated heparin (UFH), low molecular weight heparin (LMW), xylan, dextran sulfate, and glycosaminoglycans selected from heparan sulfate and chondroitin sulfate A, B, C, D, or E.

4. Polyclonal antiserum according to claim 2, wherein a) provides for 0.5-1.5 mg of antigen/injection.

5. Polyclonal antiserum according to claim 2, wherein said mammalian is a rabbit.

6. Polyclonal antiserum according to claim 2, wherein in step a), 5-10 subsequent injections, for a maximum period of 100-110 days are performed.

7. Polyclonal antiserum according to claim 2, wherein in step a), injections subsequent to the first one are performed at intervals of 4-40 days.

8. Polyclonal antiserum according to claim 2, wherein in step b), the polyclonal antiserum is collected from the mammalian after at least 100-110 days.

9. ELISA method for identifying and/or quantifying xylan sulfate levels in biological fluids with the polyclonal antiserum according to claim 1.

10. Direct or non-competitive ELISA method for the identification and/or quantification of xylan sulfate in biological fluids, characterized by the following steps:
a. immobilization of the xylan sulfate present in the biological sample, or in titrated standard solutions, on a pre-treated solid support, through incubation for 12-20 hours at room temperature;
b. blocking the aspecific binding sites by incubation with PBS containing 10% FBS for 60 minutes at room temperature;
c. incubating with polyclonal antiserum according to claim 1;
d. incubating with a rabbit IgG-specific antibody conjugated to an enzyme for 90 minutes at room temperature;
e. incubating with a solution containing the enzymatic substrate for 5-10 minutes at room temperature;
f. blocking the enzymatic reaction by addition of a denaturing solution;
g. measuring the optical density of the solution; and
h. calculating xylan sulfate concentration in the biological samples.

11. Competitive ELISA method for the identification and/or quantification of xylan sulfate levels in biological fluids, characterized by the following steps:
a. immobilizing of xylan sulfate on a pre-treated solid support, through incubation for 12-20 hours at room temperature;
b. blocking the aspecific binding sites by incubating with PBS+10% FBS for 60 minutes at room temperature;
c. incubating with both the polyclonal antiserum according to claim 1, and with the biological sample or with titrated solutions of xylan sulfate, for 90 minutes at room temperature;
d. incubating with a rabbit IgG-specific antibody conjugated to an enzyme for 90 minutes at room temperature;
e. incubating incubation with a solution containing the enzymatic substrate for 5-10 minutes at room temperature;
f. blocking the enzymatic reaction by addition of a denaturing solution;
g. measuring the optical density of the solution; and
h. calculating xylan sulfate concentration in the biological samples.

12. ELISA method according to claim 11, characterized in that the color intensity developed in step g) is inversely proportional to xylan sulfate amount.

13. ELISA method according to claim 11, characterized in that the polyclonal antiserum used in step c) is used at fixed concentration.

14. The polyclonal antiserum according to claim 2, wherein step a) provides for 1 mg of antigen/injection.

15. Polyclonal antiserum according to claim 2, wherein in step a), 5-10 subsequent injections, for a maximum period of 104 days are performed.

16. Polyclonal antiserum according to claim 2, wherein in step a), injections subsequent to the first one are performed at intervals of from 7 to 14 days.

17. Polyclonal antiserum according to claim 2, wherein in step b), the polyclonal antiserum is collected from the mammalian after 104 days.

18. The method according to claim 10, wherein said denaturing solution is 2N sulfuric acid.

19. The method according claim 11, wherein said denaturing solution is 2N sulfuric acid.

20. The method according to claim 13, wherein the polyclonal antiserum used in step c) is at a dilution 1:10000.
